(19) United States
(12) Patent Application Publication
(10) Pub. No.: US 2012/0053465 A1
(43) Pub. Date: Mar. 1, 2012

(54) ULTRASOUND DIAGNOSTIC APPARATUS AND ULTRASOUND DIAGNOSTIC METHOD

(75) Inventor: Yoshimitsu KUDOH, Ashigara-kami-gun (JP)

(73) Assignee: FUJIFILM CORPORATION, Tokyo (JP)

(21) Appl. No.: 13/211,789

(22) Filed: Aug. 17, 2011

(30) Foreign Application Priority Data
Aug. 31, 2010 (JP) 2010-194917

(51) Int. Cl. A61B 8/14

(52) U.S. Cl. 600/443

(57) ABSTRACT

An ultrasound diagnostic apparatus comprises: a diagnosis judging unit that judges whether diagnosis using an ultrasound image generated by an image producer is in progress; and a control unit that controls a transmission actuator and reception signal processors to select a high image quality mode for operation where transmission and reception of ultrasonic waves from a given number or more of oscillators of an oscillator array are performed in a whole area of the ultrasound image when the diagnosis judging unit determines that diagnosis is in progress and controls the transmission actuator and the reception signal processors to select a power saving mode for operation where transmission and reception of ultrasonic waves from at least a part of the given number or more of the oscillators of the oscillator array are stopped according to a region of the ultrasound image when the diagnosis judging unit determines that diagnosis is not in progress.

### BACKGROUND OF THE INVENTION

The present invention relates to an ultrasound diagnostic apparatus and an ultrasound diagnostic method and particularly to reduction in electric power consumption by an ultrasound diagnostic apparatus for giving a diagnosis based on an ultrasound image generated by transmission and reception of ultrasonic waves from an oscillator array of an ultrasound probe.

Conventionally, ultrasound diagnostic apparatus using an ultrasound images have been put to use in the medical field. In general, this type of ultrasound diagnostic apparatus comprises an ultrasound probe having a built-in oscillator array and an apparatus body connected to the ultrasound probe. The ultrasound probe transmits ultrasonic waves toward a subject, receives ultrasonic echoes from the subject, and the apparatus body electrically processes the reception signals to generate an ultrasound image.

In recent years, there have been developed portable ultrasound diagnostic apparatus that can be brought to a bed or transported to a site for emergency medical care. This type of ultrasound diagnostic apparatus uses a battery for power supply, and, therefore, the power consumption by the apparatus greatly affects the time length of continuous use. Since the amount of heat generated inside the apparatus increases with the power consumption, the dimensions of the apparatus needs to be increased as a measure to release heat, reducing the benefit of portability.

A great reduction in power consumption is demanded in particular in transmission and reception circuits used in, for example, a wireless probe connected wirelessly to the apparatus body because these circuits for transmitting ultrasonic waves from the oscillators and receiving ultrasound echoes need to be housed in a compact probe.

However, while a typical ultrasound diagnostic apparatus uses a voltage of 50 V to 100 V to actuate the oscillators, the voltage used to actuate the oscillators in the above-mentioned wireless probe, for example, is low because of a limited mounting space, so that the S/N ratio of the reception circuit is required to be raised in order to obtain a high image quality. In general, there is a relationship between the S/N ratio of the reception circuit and its power consumption such that reducing the power consumption while maintaining a high S/N ratio is currently difficult.

As to power saving in an ultrasound diagnostic apparatus, JP 2003-175035 A, for example, describes an apparatus that selectively stops or limits operations of an unnecessary unit among a transmitter, a receiver, a luminance processor, a memory, and the like constituting the ultrasound diagnostic apparatus according to the operation mode of the image display. JP 2009-148424 A proposes an apparatus that stops the operations of a transmission circuit or a reception circuit in a given period such as a freeze image display period, a blanking period, etc.

However, JP 2003-175035 A does not describe reduction in power consumption by a circuit related to transmission and reception of ultrasonic waves from the oscillators, and the apparatus described therein cannot be expected to produce effects in power saving in continuous use with a B-mode image display, which is typically most frequently used in a diagnostic apparatus. Therefore, an attempt to reduce power consumption with an apparatus where the probe such as a wireless probe has the transmission and reception circuits mounted therein poses problems including the necessity to achieve reduction in size itself of the transmission and reception circuits and, hence, a greatly lowered image quality.

With the apparatus described in JP 2009-148424 A, stopping the circuits in the freeze image display period, needless to say, does not produce effects on reduction in power consumption at a time when an image is displayed in real time, and achieving a great reduction in power consumption is also difficult with the B mode image display because the blanking time of transmission and reception in an ultrasound diagnostic apparatus is as short as about 1/10 of the period during which ultrasonic echoes are received by operating the reception circuit.

### SUMMARY OF THE INVENTION

The present invention has been made to resolve such problems of the past and has an object to provide an ultrasound diagnostic apparatus and an ultrasound diagnostic method that permit reduction in power consumption without lowering an image quality required for diagnosis even with the B-mode image display.

An ultrasound diagnostic apparatus according to the present invention comprises:

an ultrasound probe having an oscillator array;

a transmission actuator for supplying actuation signals to the oscillator array of the ultrasound probe to transmit an ultrasonic beam to a subject;

reception signal processors for processing reception signals outputted from the oscillator array of the ultrasound probe having received an ultrasonic echo from the subject;

an image producer generating an ultrasound image based on processed reception signals;

diagnosis judging means that judges whether diagnosis using an ultrasound image generated by the image producer is in progress; and

control means that controls the transmission actuator and the reception signal processors to select a high image quality mode for operation where transmission and reception of ultrasonic waves from a given number or more of the oscillators of the oscillator array are performed in a whole area of the ultrasound image when the diagnosis judging means determines that diagnosis is in progress and controls the transmission actuator and the reception signal processors to select a power saving mode for operation where transmission and reception of ultrasonic waves from at least a part of the given number or more of the oscillators of the oscillator array are stopped according to a region of the ultrasound image when the diagnosis judging means determines that diagnosis is not in progress.

An ultrasound diagnostic method according to the present invention comprises the steps of:

transmitting an ultrasonic beam from an oscillator array of an ultrasound probe to a subject according to actuation signals supplied from a transmission actuator;

processing reception signals outputted from the oscillator array of the ultrasound probe having received an ultrasonic echo from the subject using reception signal processors;

generating an ultrasound image with an image producer based on processed reception signals;

judging whether diagnosis using an ultrasound image generated by the image producer is in progress; and

controlling the transmission actuator and the reception signal processors to select a high image quality mode for operation where transmission and reception of ultrasonic waves from a given number or more of oscillators of the oscillator array are performed in a whole area of the ultrasound image when a judgment is made that diagnosis is in progress and controlling the transmission actuator and the reception signal processors to select a power saving mode for operation where transmission and reception of ultrasonic waves from at least a part of the given number or more of the oscillators of the oscillator array according to a region of the ultrasound image are stopped when the diagnosis judging means determines that diagnosis is not in progress.

### BRIEF DESCRIPTION OF THE DRAWINGS

FIG. 1 is a block diagram illustrating a configuration of the ultrasound diagnostic apparatus according to Embodiment 1 of the invention.

FIGS. 2A and 2B illustrate transmission and reception of the ultrasonic waves from an oscillator array according to Embodiment 1 in a high image quality mode and a power saving mode, respectively.

FIG. 3 is a timing chart illustrating the operation of Embodiment 1.

FIGS. 4A and 4B illustrate transmission and reception of the ultrasonic waves from an oscillator array according to Embodiment 2 in a high image quality mode and a power saving mode, respectively.

FIGS. 5A and 5B illustrate a shift in an ultrasound image due to a movement of an ultrasound probe and a shift in an ultrasound image due to a movement in a subject in Embodiment 3, respectively.

### DETAILED DESCRIPTION OF THE INVENTION

Embodiments of the present invention will be described below based on the appended drawings.

### Embodiment 1

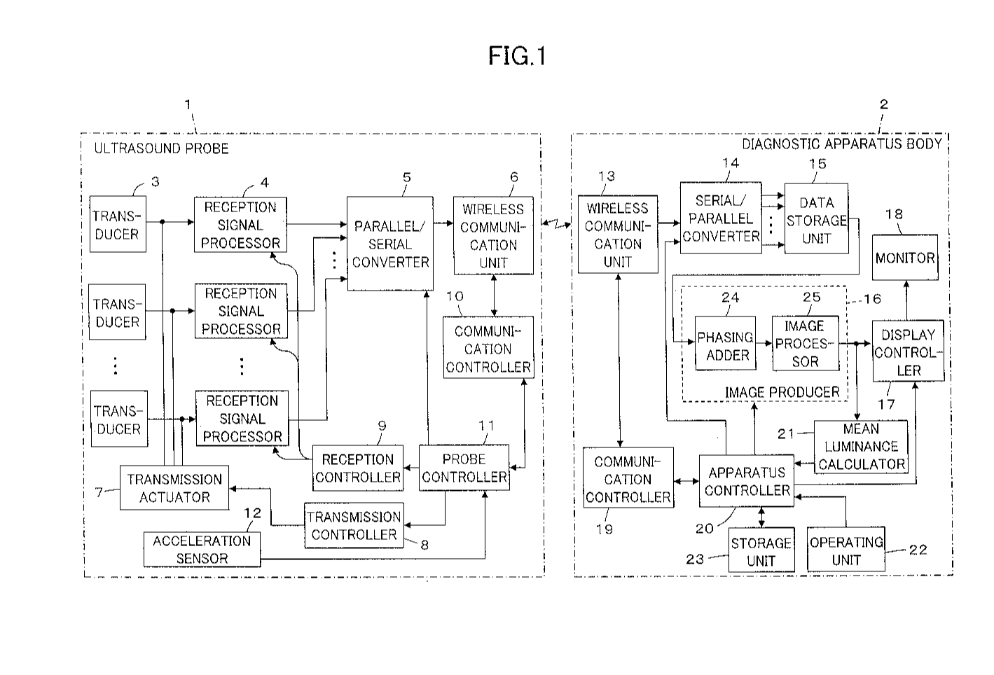

FIG. 1 illustrates a configuration of the ultrasound diagnostic apparatus according to Embodiment 1 of the invention. The ultrasound diagnostic apparatus comprises an ultrasound probe 1 and a diagnostic apparatus body 2 that is connected to the ultrasound probe 1 via wireless communication.

The ultrasound probe 1 comprises a plurality of ultrasound transducers 3 constituting a unidimensional or two-dimensional oscillator array, and the transducers 3 are connected to reception signal processors 4, which in turn are connected to a wireless communication unit 6 via a parallel/serial converter 5. The transducers 3 are connected to a transmission controller 8 via a transmission actuator 7, and the reception signal processors 4 are connected to a reception controller 9, while the wireless communication unit 6 is connected to a communication controller 10. The parallel/serial converter 5, the transmission controller 8, the reception controller 9, and the communication controller 10 are connected to a probe controller 11. The ultrasound probe 1 has a built-in acceleration sensor 12 for detecting a movement of the ultrasound probe 1, and the acceleration sensor 12 is connected to the probe controller 11.

The transducers 3 each transmit ultrasonic waves according to actuation signals supplied from the transmission actuator 7 and receive ultrasonic echoes from the subject to output reception signals. Each of the transducers 3 is composed of an oscillator comprising, for example, a piezoelectric body such as a piezoelectric ceramic represented by a PZT (titanate zirconate lead), a polymeric piezoelectric device represented by a PVDF (polyvinylidene flouride), and the like and electrodes each provided on both ends of the piezoelectric body.

When the electrodes of each of such oscillators are supplied with a voltage, which may be in the form of pulse or continuous waves, the piezoelectric body expands and contracts and the oscillator generates ultrasonic waves in the form of pulse or continuous waves. These ultrasonic waves are synthesized to form an ultrasonic beam. As each oscillator receives propagating ultrasonic waves, it expands and contracts to generate an electric signal and outputs the electric signal as reception signal of the ultrasonic waves.

The transmission actuator 7 comprises, for example, a plurality of pulsers and adjusts the delay amounts of actuation signals for the respective transducers 3 based on a transmission delay pattern selected by the transmission controller 8 so that the ultrasonic waves transmitted from the transducers 3 form a broad ultrasonic beam to cover an area of a tissue of the subject, supplying the transducers 3 with the adjusted actuation signals.

Under the control of the reception controller 9, the reception signal processor 4 on each channel subjects the reception signal outputted from the corresponding transducer 3 to quadrature detection or quadrature sampling process to produce a complex base band signal, samples the complex base band signal to generate sample data containing information on the area of the tissue, and supplies the parallel/serial converter 5 with the sample data. The reception signal processors 4 may generate sample data by performing data compression for high-efficiency coding on the data obtained by sampling the complex base band signals.

The parallel/serial converter 5 converts parallel sample data generated by reception signal processors 4 on the plurality of channels into serial sample data.

The wireless communication unit 6 performs carrier modulation according to the serial sample data to generate a transmission signal and supplies an antenna with the transmission signal so that the antenna transmits radio waves to achieve transmission of the sample data. The modulation methods that may be employed herein include ASK (Amplitude Shift Keying), PSK (Phase Shift Keying), QPSK (Quadrature Phase Shift Keying), and 16QAM (16 Quadrature Amplitude Modulation).

The wireless communication unit 6 transmits the sample data to the diagnostic apparatus body 2 through wireless communication with the diagnostic apparatus body 2, receives various control signals from the diagnostic apparatus body 2, and outputs the received control signals to the communication controller 10. The communication controller 10 controls the wireless communication unit 6 so that the sample data is transmitted with a transmission wave intensity that is set by the probe controller 11 and outputs various control signals received by the wireless communication unit 6 to the probe controller 11.

The acceleration sensor 12 detects the acceleration generated with the movement of the ultrasound probe 1 and outputs the detected acceleration to the probe controller 11.

The probe controller 11 controls various components of the ultrasound probe 1 according to control signals transmitted from the diagnostic apparatus body 2 and the output signal from the acceleration sensor 12.

The ultrasound probe 1 has a built-in battery, not shown, which supplies power to the circuits inside the ultrasound probe 1.

The ultrasound probe 1 may be an external type probe such as linear scan type, convex scan type, and sector scan type or a probe of, for example, a radial scan type used for an ultrasound endoscope.

On the other hand, the diagnostic apparatus body 2 comprises a wireless communication unit 13, which is connected to a data storage unit 15 via a serial/parallel converter 14. The data storage unit 15 is connected to an image producer 16. The image producer 16 is connected to a monitor 18 via a display controller 17. The wireless communication unit 13 is also connected to a communication controller 19; the serial/parallel converter 14, the image producer 16, the display controller 17, and the communication controller 19 are connected to an apparatus controller 20. The image producer 16 is connected to a mean luminance calculator 21, which in turn is connected to the apparatus controller 20. The apparatus controller 20 is connected to an operating unit 22 for an operator to perform input operations and a storage unit 23 for storing operation programs.

The wireless communication unit 13 performs wireless communication with the ultrasound probe 1 to transmit various control signals to the ultrasound probe 1. The wireless communication unit 13 demodulates the signal received by the antenna to output serial sample data.

The communication controller 19 controls the wireless communication unit 13 so that the various control signals are transmitted with a transmission radio wave intensity that is set by the apparatus controller 20.

The serial/parallel converter 14 converts the serial sample data outputted from the wireless communication unit 13 into parallel sample data. The data storage unit 15 is configured by a memory, a hard disk, or the like and stores at least one frame of sample data converted by the serial/parallel converter 14.

The image producer 16 performs reception focusing process on every frame of sample data read out from the data storage unit 15 to generate an image signal representing an ultrasound diagnostic image. The image producer 16 comprises a phasing adder 24 and an image processor 25.

The phasing adder 24 selects one reception delay pattern from the plurality of previously stored reception delay patterns according to the reception direction set in the apparatus controller 20 and, based on that selected reception delay pattern, provides the complex base band signals represented by the sample data with respective delays before adding them to perform the reception focusing process. By this reception focusing, a base band signal (sound ray signal) where the ultrasonic echoes are well focused is generated.

The image processor 25 generates a B-mode image signal, which is tomographic image information on a tissue inside the subject, according to the sound ray signal generated by the phasing adder 24. The image processor 25 comprises an STC (sensitivity time control) and a DSC (digital scan converter). For the sound ray signal, the STC corrects attenuation due to distance according to the depth of the reflection position of the ultrasonic waves. The DSC converts the sound ray signal corrected by the STC into an image signal (raster conversion) compatible with the scanning method of an ordinary television signal and performs required image processing, such as contrast processing, to generate a B mode image signal.

The display controller 17 causes the monitor 18 to display an ultrasound diagnostic image according to the image signal generated by the image producer 16. The display unit 18 comprises a display device such as an LCD, for example, and displays an ultrasound diagnostic image under the control of the display controller 17.

The mean luminance calculator 21 calculates a mean luminance value of at least a part of the ultrasound image in the preceding frame generated by the image producer 16. For example, the mean luminance value may be a mean luminance value for the whole area of the ultrasound image or a mean luminance value for a part of the ultrasound image such as a central region.

The apparatus controller 20 makes a judgment as to whether a diagnosis is in progress using an ultrasound image generated by the image producer 16 according to the output signal from the acceleration sensor 12 of the ultrasound probe 1 and the mean luminance value of the ultrasound image in the preceding frame and selects one of the high image quality mode and the power saving mode according to the judgment made. More specifically, the apparatus controller 20 judges that a diagnosis using the ultrasound image is in progress when the ultrasound probe 1 is judged not to be moving based on the output signal from the acceleration sensor 12 and the mean luminance value obtained by the mean luminance calculator 21 exceeds a given threshold, and controls the reception signal processors 4 and the transmission actuator 7 of the ultrasound probe 1 to operate in the high image quality mode. On the other hand, when the ultrasound probe 1 is judged to be moving based on the output signal from the acceleration sensor 12 or when the mean luminance value obtained by the mean luminance calculator 21 does not exceed a given threshold, the apparatus controller 20 judges that a diagnosis using the ultrasound image is not in progress and controls the reception signal processors 4 and the transmission actuator 7 to operate in the power saving mode.

In such diagnostic apparatus body 2, while the serial/parallel converter 14, the image producer 16, the display controller 17, the communication controller 19, and the apparatus controller 20 are each constituted by a CPU and an operation program for causing the CPU to perform various kinds of processing, they may be constituted by a digital circuit. The aforementioned operation program is stored in the storage unit 23. The recording medium in the storage unit 23 may be a flexible disk, MO, MT, RAM, CD-ROM, DVD-ROM or the like besides a built-in hard disk.

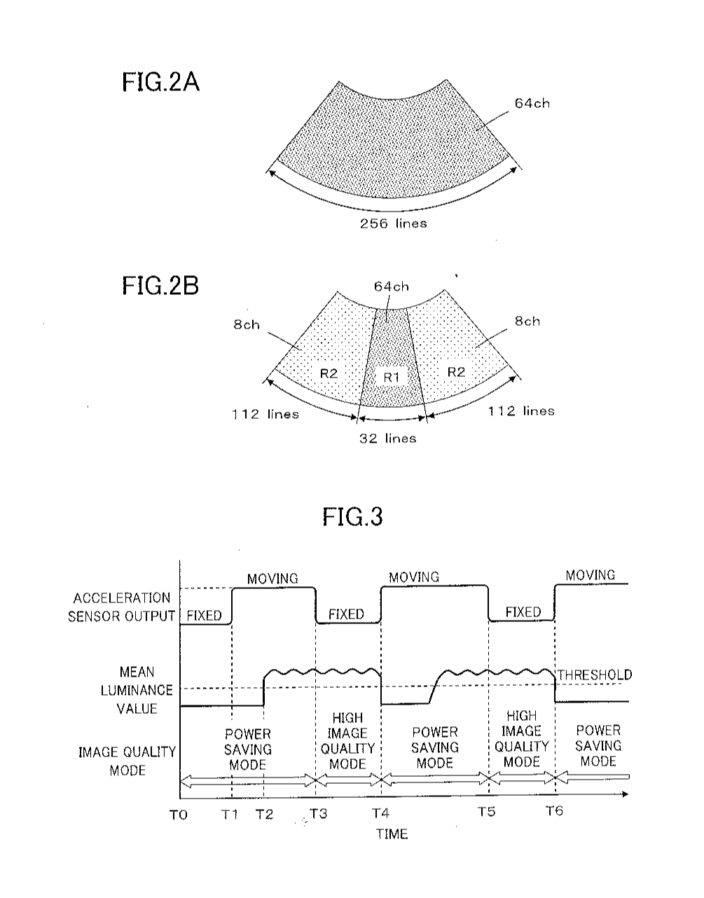

Now, the high image quality mode and the power saving mode will be described referring to FIGS. 2A and 2B.

In the high image quality mode, a given number or more of the transducers 3 constituting the oscillator array are operated for the whole area of the screen, i.e., for all the scan lines, to transmit and receive ultrasonic waves. As illustrated in FIG. 2A, for example, when the oscillator array has 64 channels of transducers 3 and the screen has 256 scan lines, the transmission controller 8 controls the transmission actuator 7 to operate a given number or more of the transducers 3 from among the 64 channels for each of the 256 scan lines, while the reception controller 9 controls a given number or more of the reception signal processors 4 from among the 64 channels. An image having a high image quality may be thus obtained.

In the power saving mode, on the other hand, a given number or more of the transducers 3 constituting the oscillator array for the scan lines located in a central region of the screen are operated to transmit and receive ultrasonic waves as in the high image quality mode, while for the scan lines located in the other regions of the screen than the central region, transmission and reception of ultrasonic waves from some of the given number or more of the transducers 3 are stopped unlike in the high image quality mode where the given number or more of the transducers 3 are operated, so that fewer transducers 3 are operated than in the high image quality mode for transmission and reception of the ultrasonic waves. Where, for example, the oscillator array has 64 channels of the transducer array and the screen has 256 scan lines as illustrated in FIG. 2B, the transmission controller 8 controls the transmission actuator 7 and the reception controller 9 controls the reception signal processors 4 so that a given number or more of the transducers 3 from among the 64 channels are operated for the 32 scan lines located in a region R1 in a central part of the screen, whereas for the 112+112=224 scan lines located in the other regions R2 than the region R1, i.e., on both sides of the region R1 in the screen, only 8 channels, which is fewer than a given number, of the transducers 3 of the 64 channels, are operated, while the operations of the remaining 56 channels of the transducers 3 are stopped. Thus, power saving can be achieved.

A “given number or more of transducer 3” operated in the high image quality mode means a number of transducers 3 that can be processed in parallel at the same time, i.e., a number equal to or smaller than the number of the reception signal processors 4 provided in the ultrasound probe 1 and greater than the number of transducers operated in the power saving mode.

Next, the operation of Embodiment 1 will be described.

When ultrasound diagnosis starts, the transducers 3 transmit ultrasonic waves according to the actuation signals supplied from the transmission actuator 7, and the reception signals outputted from the transducers 3 that have received the ultrasonic echoes from the subject are supplied to the corresponding reception signal processors 4 to generate sample data, which undergoes conversion into serial data by the parallel/serial converter 5 and then are transmitted wirelessly from the wireless communication unit 6 to the diagnostic apparatus body 2. The sample data received by the wireless communication unit 13 of the diagnostic apparatus body 2 is converted into parallel data through the serial/parallel converter 14 and stored in the data storage unit 15. Further, the data storage unit 15 reads out the sample data by frame, and the image producer 16 generates the image signal and, based on this image signal, the display controller 17 controls the monitor 18 to display the ultrasound diagnostic image.

Thus, the mean luminance value of at least a part of the ultrasound image generated by the image producer 16 is calculated by the mean luminance calculator 21 of the diagnostic apparatus body 2 and entered in the apparatus controller 20. The apparatus controller 20 judges based on this mean luminance value whether acquisition of the ultrasound diagnostic image is in progress. Where the ultrasound image is not being generated when the traducers 3 have transmitted ultrasonic waves in a blanking period, for example, or where the ultrasound probe 1 is left out of contact with the surface of the subject, then the mean luminance value calculated by the mean luminance calculator 21 does not exceed a given threshold, and, therefore, a judgment can be made that acquisition of the ultrasound diagnostic image is not in progress.

The acceleration sensor 12 detects the acceleration generated with the movement of the ultrasound probe 1 and outputs the detected data to the probe controller 11. The detected data is transmitted from the probe controller 11 to the wireless communication unit 6 via the communication controller 10 and wirelessly transmitted from the wireless communication unit 6 to the diagnostic apparatus body 2. The detected data received by the wireless communication unit 13 of the diagnostic apparatus body 2 is entered in the apparatus controller 20 via the communication controller 19, whereupon the apparatus controller 20 judges whether the ultrasound probe 1 is being moved or fixed in position based on the detected data.

Then, the apparatus controller 20 judges that a diagnosis using the ultrasound image is in progress when the ultrasound probe 1 is judged not to be moving based on the output signal from the acceleration sensor 12 and the mean luminance value obtained by the mean luminance calculator 21 exceeds a given threshold, and selects the high image quality mode. On the other hand, when a judgment is made that the ultrasound probe 1 is moving based on the output signal from the acceleration sensor 12 or when the mean luminance value obtained by the mean luminance calculator 21 does not exceed a given threshold, the apparatus controller 20 judges that a diagnosis using the ultrasound image is not in progress and selects the power saving mode.

For example, in the timing chart of FIG. 3, when the ultrasound probe 1 is fixed in position but the mean luminance value does not exceed the threshold as in time T0 to T1, when the ultrasound probe 1 is moving and the mean luminance value does not exceed the threshold as in time T1 to T2, and when the mean luminance value is above the threshold but the ultrasound probe 1 is moving as in time T2 to T3, the apparatus controller 20 judges that a diagnosis using the ultrasound image is not in progress and selects the power saving mode. That is, the transmission actuator 7 and the reception signal processors 4 are controlled via the probe controller 11 of the ultrasound probe 1 so that transmission and reception of the ultrasonic waves from a part of the transducers 3 are stopped, and only the remaining transducers 3, fewer than in the high image quality mode, are operated to transmit and receive the ultrasonic waves.

On the other hand, when the ultrasound probe 1 is fixed in position and the mean luminance value is above the threshold as in time T3 to T4, the apparatus controller 20 judges that a diagnosis using the ultrasound image is in progress and selects the high image quality mode. That is, the transmission actuator 7 and the reception signal processors 4 are controlled via the probe controller 11 of the ultrasound probe 1 so that a given number or more of the transducers 3 are operated from among the transducers 3 constituting the oscillator array to transmit and receive the ultrasonic waves.

Likewise, in time T4 to T5 and in time T6 onward, where the ultrasound probe 1 is moving, the power saving mode is selected, while in Time T5 to T6, where the ultrasound probe is fixed in position and the mean luminance value is above the threshold, the high image quality mode is selected.

Thus, a judgment is made as to whether a diagnosis using the ultrasound image is in progress based on the output signal from the acceleration sensor 12 and the mean luminance value obtained by the mean luminance calculator 21 to control the transmission actuator 7 and the reception signal processors 4 of the ultrasound probe 1 so that when a judgment is made that the diagnosis is in progress, the operation is performed in the high image quality mode whereas when a judgment is made that the diagnosis is not in progress, the operation is performed in the power saving mode. As a result, a high quality image can be displayed only when the operator is giving a diagnosis using a displayed image, and, when not, the mode can be automatically switched to the power saving mode. Therefore, even with the B-mode image display, which is frequently used, power saving can be achieved without reducing an image quality as required for a diagnosis or affecting the diagnosis.

### Embodiment 2

While the power saving mode according to Embodiment 1 is such that transmission and reception of ultrasonic waves from a part of the transducers 3 of the oscillator array are stopped for the regions R2 other than the central region of the ultrasound image, the invention is not limited this way and power saving may be effected selectively according to the frame.

According to Embodiment 2, in the high image quality mode, ultrasonic waves are transmitted and received from a given number or more of the transducers 3 for all the frames in the whole area of the ultrasound image similarly to Embodiment 1. In the power saving mode, on the other hand, ultrasonic waves from the oscillator array are transmit and receive for all the frames in the central region of the ultrasound image while in the other regions than the central region of the ultrasound image, transmission and reception of ultrasonic waves from the oscillator array are stopped selectively according to the frame. Then, the image producer 16 is caused to generate the ultrasound image by superposing images of the regions other than the central region of the frame for which ultrasonic waves from the oscillator array were transmitted and received in the whole area of the ultrasound image over the regions other than the central region of the ultrasound image for which transmission and reception of ultrasonic waves from the oscillator array were stopped.

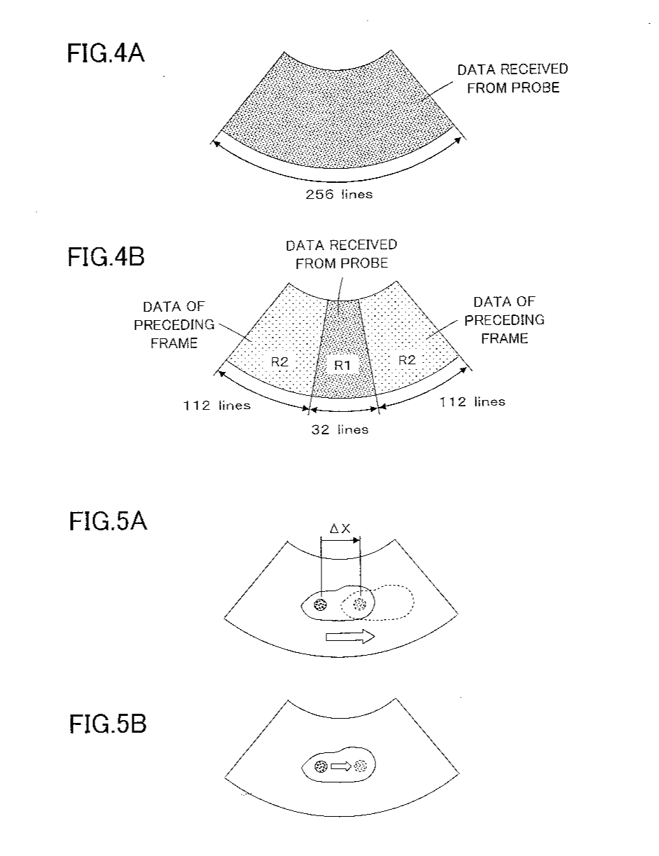

When, for example, the screen has 256 scan lines as illustrated in FIG. 4A, a given number or more of the transducers 3 are operated from among the transducers 3 of the oscillator array for all the frames to transmit and receive ultrasonic waves in the whole area of the ultrasound image, i.e., in all of the 256 scan lines in the high image quality mode. An image having a high image quality may be thus obtained.

Further, in the power saving mode, transmission and reception of ultrasonic waves from the oscillator array are varied selectively according to the frame. That is, in a frame, a given number or more of the transducers 3 from among the transducers 3 of the oscillator array are operated to transmit and receive ultrasonic waves for all of the 256 scan lines as in the high image quality mode. In the next frame, ultrasonic waves from the oscillator array are transmitted and received for the 32 scan lines in the central region R1 of the ultrasound image, while transmission and reception of ultrasonic waves from the oscillator array are stopped for 112+112=224 lines in the regions R2 other than the region R1 located on both sides of the region R1 in the screen. Then, the ultrasound image is generated with the region R2 of the image of the preceding frame superposed over the regions R2 located on both sides in the screen as illustrated in FIG. 4B. Thus, power saving can be achieved.

In the power saving mode, transmission and reception of ultrasonic waves from the oscillator array in the regions R2 on both sides in the screen may be stopped for every frame or may be stopped for several consecutive frames and then resumed for the next frame.

### Embodiment 3

While a judgment is made as to whether the ultrasound probe 1 is moving or fixed in position based on the output signal from the acceleration sensor 12 in Embodiments 1 and 2, the invention is not limited thereto. As illustrated in FIG. 5A, for example, the apparatus controller 20 of the diagnostic apparatus body 2 may be configured to detect the movement of the ultrasound probe 1 based on a shift ΔX in the ultrasound image between frames.

The apparatus controller 20 makes a judgment as to whether a diagnosis is in progress using the ultrasound image generated by the image producer 16 based on the shift ΔX in the ultrasound image between frames and the mean luminance value of the preceding frame of the ultrasound image obtained by the mean luminance calculator 21 and selects either the high image quality mode or the power saving mode according to the judgment made.

In this case, it is preferable to determine using data showing the Doppler effect whether the shift in the ultrasound image between frames is due to a movement of the ultrasound probe 1 as illustrated in FIG. 5A or due to a movement in the subject as illustrated in FIG. 5B. Even when there is a shift in the ultrasound image between frames and the shift is due to a movement in the subject, the ultrasound probe 1 itself may be judged not to be moving but to be fixed in position. Judgment as to such shift may be made by the apparatus controller 20 of the diagnostic apparatus body 2.

While when, in Embodiments 1 to 3, the ultrasound probe 1 is judged not to be moving and the mean luminance value of the ultrasound image of the preceding frame exceeds a given threshold, a judgment is made that diagnosis using the ultrasound image is in progress and the high image quality mode is selected, the high image quality mode may also be selected even when the ultrasound probe 1 is not fixed in position if the movement speed of the ultrasound probe 1 becomes a given value or smaller. This is because a case is conceivable where, depending on the manner in which the operator gives a diagnosis, the whole image is observed with a high image quality when the ultrasound probe 1 is being moved at a low speed on the surface of the subject, while when the ultrasound probe 1 is fixed in position to focus the observation, a central region of the screen need only be carefully watched. The movement speed of the ultrasound probe 1 at which the mode is switched between the high image quality mode and the power saving mode is preferably selected and set as appropriate by the operator.

Further, the ultrasound probe 1 or the diagnostic apparatus body 2 may be provided with a still image acquisition switch to enable acquisition of a still image when the still image acquisition switch is operated. For the operator to operate the still image acquisition switch to acquire a still image when the apparatus is in the power saving mode, it is preferable that a given number or more of the transducers 3 are operated in the whole area of the image to acquire a still image having a high image quality, whether the ultrasound probe 1 is moving or fixed in position. While the acquired still image is being displayed, transmission and reception of ultrasonic waves from the oscillator array may be stopped.

The number of channels and scan lines in the above embodiments are only illustrative examples and may be changed as appropriate.

Although the ultrasound probe 1 and the diagnostic apparatus body 2 are connected to each other by wireless communication in Embodiments 1 to 3, the invention is not limited thereto and the ultrasound probe 1 may be connected to the diagnostic apparatus body 2 via a connection cable. Such configuration obviates the necessity to provide the wireless communication unit 6 and the communication controller 10 of the ultrasound probe 1, the wireless communication unit 13 and the communication controller 19 of the diagnostic apparatus body 2, and the like.

What is claimed is:

1. An ultrasound diagnostic apparatus comprising:
an ultrasound probe having an oscillator array;
a transmission actuator for supplying actuation signals to the oscillator array of the ultrasound probe to transmit an ultrasonic beam to a subject;
reception signal processors for processing reception signals outputted from the oscillator array of the ultrasound probe having received an ultrasonic echo from the subject;
an image producer generating an ultrasound image based on processed reception signals;
diagnosis judging means that judges whether diagnosis using an ultrasound image generated by the image producer is in progress; and
control means that controls the transmission actuator and the reception signal processors to select a high image quality mode for operation where transmission and reception of ultrasonic waves from a given number or more of the oscillators of the oscillator array are performed in a whole area of the ultrasound image when the diagnosis judging means determines that diagnosis is in progress and controls the transmission actuator and the reception signal processors to select a power saving mode for operation where transmission and reception of ultrasonic waves from at least a part of the given number or more of the oscillators of the oscillator array are stopped according to a region of the ultrasound image when the diagnosis judging means determines that diagnosis is not in progress.

2. The ultrasound diagnostic apparatus according to claim 1, wherein the control means controls the transmission actuator and the reception signal processors so that transmission and reception of ultrasonic waves from the part of the oscillators of the oscillator array are stopped in regions other than a central region of the ultrasound image in the power saving mode.

3. The ultrasound diagnostic apparatus according to claim 1, wherein the control means controls the transmission actuator and the reception signal processors so that transmission and reception of ultrasonic waves from the oscillator array are stopped in the regions other than the central region of the ultrasound image selectively according to a frame in the power saving mode, and causes the image producer to generate an ultrasound image such that images of the regions other than the central region of the frame for which transmission and reception of ultrasonic waves from the oscillator array were effected in a whole area of the ultrasound image are superposed over the other regions than the central region of the ultrasound image for which transmission and reception of ultrasonic waves from the oscillator array were stopped.

4. The ultrasound diagnostic apparatus according to claim 1, wherein the diagnosis judging means comprises:
movement detecting means that detects a movement of the ultrasound probe; and
mean luminance calculator for calculating a mean luminance value of at least a part of the ultrasound image generated by the image producer,
the diagnosis judging means judging whether diagnosis is in progress using an ultrasound image generated by the image producer based on a detection result obtained by the movement detecting means and a mean luminance value obtained by the mean luminance calculator.

5. The ultrasound diagnostic apparatus according to claim 4, wherein the movement detecting means is an acceleration sensor.

6. The ultrasound diagnostic apparatus according to claim 4, wherein the movement detecting means detects a movement of the ultrasound probe based on a shift in an ultrasound image between frames.

7. The ultrasound diagnostic apparatus according to claim 6, further comprising shift judging means that judges whether a shift in an ultrasound image between frames is due to a movement of an ultrasound probe or a movement in a subject using data showing Doppler effect.

8. An ultrasound diagnostic method comprising the steps of:
transmitting an ultrasonic beam from an oscillator array of an ultrasound probe to a subject according to actuation signals supplied from a transmission actuator;
processing reception signals outputted from the oscillator array of the ultrasound probe having received an ultrasonic echo from the subject using reception signal processors;
generating an ultrasound image with an image producer based on processed reception signals;
judging whether diagnosis using an ultrasound image generated by the image producer is in progress; and
controlling the transmission actuator and the reception signal processors to select a high image quality mode for operation where transmission and reception of ultrasonic waves from a given number or more of oscillators of the oscillator array are performed in a whole area of the ultrasound image when a judgment is made that diagnosis is in progress and controlling the transmission actuator and the reception signal processors to select a power saving mode for operation where transmission and reception of ultrasonic waves from at least a part of the given number or more of the oscillators of the oscillator array according to a region of the ultrasound image are stopped when the diagnosis judging means determines that diagnosis is not in progress.

9. The ultrasound diagnostic method according to claim 8, wherein the transmission actuator and the reception signal processors are so controlled that transmission and reception of ultrasonic waves from the part of the oscillators of the oscillator array are stopped for regions other than a central region of the ultrasound image in the power saving mode.

10. The ultrasound diagnostic method according to claim 8, wherein the transmission actuator and the reception signal processors are so controlled that transmission and reception of ultrasonic waves from the oscillator array are stopped for regions other than a central region of the ultrasound image selectively according to a frame in the power saving mode to generate an ultrasound image such that images of the regions other than the central region of the frame for which transmission and reception of ultrasonic waves from the oscillator array were effected in a whole area of the ultrasound image are superposed over the other regions than the central region of the ultrasound image for which transmission and reception of ultrasonic waves from the oscillator array were stopped.

11. The ultrasound diagnostic method according to claims 8, further comprising the steps of:
judging whether the ultrasound probe is moving or not; and
calculating a mean luminance value of at least a part of the ultrasound image generated by the image producer,
the judgment whether a diagnosis using an ultrasound image is in progress being made based on the judgment as to whether the ultrasound probe is moving or not and the calculated mean luminance value.

12. The ultrasound diagnostic method according to claim 11, wherein whether the ultrasound probe is moving or not is determined by an acceleration sensor.

13. The ultrasound diagnostic method according to claim 11, wherein whether the ultrasound probe is moving or not is determined based on a shift in the ultrasound image between frames.

14. The ultrasound diagnostic apparatus according to claim 13, wherein data showing Doppler effect is used to determine whether a shift in an ultrasound image between frames is due to a movement of an ultrasound probe or a movement in a subject.
